(19) United States
(12) Patent Application Publication
(10) Pub. No.: US 2017/0080120 A1
(43) Pub. Date: Mar. 23, 2017

(54) PHASE-SHIFTING FORMULATIONS

(71) Applicant: ULTRAST, INC, Oceanside, NY (US)

(72) Inventors: Howard L. LEVINE, Oceanside, NY (US); Donimique DE ZIEGLER, Paris (FR)

(73) Assignee: ULSTRAST, INC., Oceanside, NY (US)

(21) Appl. No.: 15/311,087

(22) PCT Filed: May 15, 2015
(86) PCT No.: PCT/US2015/031012

§ 371 Date: Nov. 14, 2016

Related U.S. Application Data
(60) Provisional application No. 61/994,491, filed on May 16, 2014.

(51) Int. Cl. A61L 26/00

(52) U.S. Cl. CPC A61L 26/008; A61L 26/0066; A61L 26/0023; A61L 2300/404; A61L 2300/41

(57) ABSTRACT

The inventive composition first is highly viscous, remaining in place when administered to a patient. Then it decreases in viscosity and liquefies, facilitating easy removal, after a period of time ranging from minutes to weeks, such as after a change in temperature or other trigger; or after another component is added to cause liquefaction. Such compositions have many different medical uses, optionally with a treating agent contained in, or held in place by, the composition, such as, without limitation, prevention or reduction in scarring or adhesions after surgery involving the uterus or other body or organ cavities or other sites, by keeping raw areas of the tissue or tissue walls separated from each other during healing; delivery or retention of treating agents in body or organ cavities or other sites of administration; protection of wounds, burns, and other injuries; and holding tissue grafts in place. Even cosmetic uses are available.

### FIELD OF INVENTION

The present invention is directed to phase shifting formulations, typically but not always in the form of a gel, with various uses that benefit from its properties of (1) remaining in place for a desired period of time at a higher viscosity before liquefying to facilitate removal or dissipation; (2) maintaining distension of a virtual body or organ cavity for a desired period of time in a higher viscosity state, before liquefying to facilitate removal or dissipation; and (3) the fact that such formulations tend not to mix with adjacent blood and other body fluids, instead tending to act as a barrier or wall that seems to act as a barrier, minimizing loss of such fluids. Treating agents and/or beneficial adjuvants and other agents may also be added to the formulation as appropriate to the desired use. One of ordinary skill in the art of preparing formulations would readily be able to prepare a suitable formulation with the desired degree and duration of initial viscosity, before liquefaction enables removal of the formulation.

One example is use as a long lasting distension product for preventing scarring, in some cases called synechiae, and all forms of adhesion or adhesive tissue reaction associated with certain uterine procedures, including surgery performed by hysteroscopy, laparoscopy or laparotomy. In a broader related context, the invention is directed to a product for similar use after procedures taking place in or on any other body or organ cavity, or on other body tissue.

Similarly, such formulations may be useful in facilitating desired scarring, such as following urological procedures. For example, in cases where tissue is excised, healing may be enhanced via initial scar formation. For all uses involved with healing/scarring/preventing adherence—each situation is different and depends upon what is needed for the use. Formulations can easily be prepared that remain in one phase, then upon addition of a second product cause a phase-shift to have the product removed easily. Current practice has the need for either repeated applications, difficult removal, or the fact that such products are unavailable due to the site of the problem in the body. This phase shifting formulation concept can protect the tissues from adhering to other tissues, or enhance healing or scarring as needed.

For example, following urological surgery a slight distension of the urethra while scarring takes place—together with the temporary by-passing of urine flow through the use of a supra-pubic catheter—may hasten and facilitate the physiological nature of that scarring and prevent undesired adhesions. In this case, the instillation of a phase-shifting formulation in gel form immediately post surgery would keep the urethra open—while urine flow is bypassed using a supra-pubic catheter—for the time needed for proper healing of the surgical scar, as for example 10-20 days.

Other uses include, for example, delivery of treating agents, beneficial coatings, or other helpful agents within body or organ cavities or otherwise to various sites in or on the body. Such delivery can be achieved and maintained during procedures such as those discussed above, or independently or repeatedly after a surgery or other procedure, or after other injury to the body tissue being treated. The invention allows maintaining contact of a treating agent or protective coating or agent with a sensitive tissue for a desired period of time, before dissipating or becoming easy to remove without significantly disturbing the tissue. This could include burns, wounds, and other injured or distressed tissues, and could be applied, for example, as an emergency protection pending full medical attention, or after medical attention has been completed, as deemed appropriate by the practitioner. The agents could be mixed into the formulation, and/or they could be added separately to provide a higher concentration directly on or near the tissue desired to be treated.

Similarly, the formulation could serve to coat or protect sensitive or damaged tissue, without the disadvantages associated with fabric coverings. For example, such formulations could help protect and even help treat burns and other open wounds; post-traumatic scar tissue of internal organs that may be encountered in gynecology (involving intra-uterine tissue or fallopian tubes), urology, or ENT. Placement in cavity is dependent on site, as would be readily known to a practitioner. Timing is flexible, as discussed elsewhere above and below.

In the uterus, surgical removal of pathologies such as fibroids that expand into the uterine cavity (sub-mucosal) or a polyp that is attached by a large base to the lining of the uterine cavity (endometrium) may cause undesired scar formation that reaches across the uterine cavity, causing a pathology—uterine synechiae—capable of interfering with fecundity. Applying the formulation after surgery is completed would keep the walls of the uterine cavity open while proper healing takes place and thus, prevent the undue scarring that often takes place when raw surfaces of opposing side of the cavity come in direct contact. After proper healing of the epithelium itself—commonly achieved in 2 weeks, especially if the patient is on estrogen—further distension of the cavity is no longer necessary. At that point the formulation is transformed into liquid and simply expelled. The formulation can be made to become liquid through different ways of controlling its phase shifting properties, such as, for example, by injecting a few CC of a second or ‘liquefying’ formulation.

Other potential medical uses for such formulations include, but are not limited to, the following: holding in place fragments of graft tissue and/or stem cell preparations, due to presence of the viscous product without physically sticking to the body or organs. In this case, a cell preparation enriched in stem cells would be gently layered upon the area needing profound reconstruction of the uterine cavity lining while being kept in place and separating the walls of the uterine cavity through application of the formulation in gel stage. Typically, it is believed that separating the walls of the uterine cavity and keeping the cell preparation in place for 2-3 weeks should suffice for letting a colony of stem cells have a chance of regenerating a new lining of the uterine cavity while being stimulated by an estrogen preparation. At that point further distension of the cavity is not deemed necessary anymore. The formulation based on its phase shifting properties would be allowed or caused to liquefy (through different means as for example by the injection through the uterine cervix of a second liquefying formulation) and be expelled naturally. For such treatment, the formulation could be accompanied by the administration any type of growth factor and/or anti-rejection agent to promote successful grafting.

As described above, such treatment applied on the cell colony and held in place by the formulation in gel state or directly integrated in the formulation itself. In either case, the duration of treatment would be in the range of 10-20 days. This corresponds to the time taken for a new epithelium to grow if not impeded by the proliferation of fibroblast that would be stimulated by the inflammation reaction resulting from raw surfaces of the uterine cavity coming into contact with each other (if not kept separated by the gel formulation).

It is believed that the formulation thus would facilitate the proper healing of raw surfaces after surgery reducing the risk of adhesion formation—thus, improving the chances for surgery to be successful—and in the case of stem cell therapy, favor the implantation of the stem cells and their ability to recolonize the damaged surface of the uterine cavity and allow the development of a regenerated endometrium.

The formulations may also be used, for example, during temporary distension of a joint to facilitate diagnosis of damage or developing lesions, such as those that are degenerative or linked to inflammation or cancer.

The instant formulations could also be used for non-medical uses. For example, such formulations may prove useful in cosmetic procedures such as protecting the scalp or facial skin during hair bleaching and coloring procedures; providing protection during tanning in the sun or tanning booths; promoting healing of the site of a fresh tattoo on the body; and maintaining contact with skin or hair for an extended period of time, such as with moisturizing or regenerative agents overnight.

### BACKGROUND OF INVENTION

U.S. Pat. No. 7,727,155 is entitled “Medium for contrast enhancement or convenience for ultrasonic, endoscopic, and other medical examinations.” That patent provides formulations for contrast interphase between normally collapsed structures, particularly in a body or organ cavity, for enhancing medical imaging procedures such as ultrasound, X-ray, CT-scan and MRI. The phase shifting properties of the formulations provide for transforming the initial gel consistency needed for generating contrast, after a sufficient period of time for the procedure, to a liquid for easy removal or expulsion. Now, the same basic formulations has been recognized to potentially provide significant benefits in many other types of medical and dental procedures, as well as other settings such as for cosmetic uses.

Preventing Post-Surgical Scarring and Other Adhesions

Surgery that involves the uterine cavity may be indicated for the removal of intra uterine pathologies that cause abnormal bleeding and/or infertility. These primarily include polyps, sub-mucosal extension of fibroids (developing inside the uterine cavity) and abnormal scar tissue or synechiae, which developed because of prior surgery, infection, or retention of the product of conception or placenta following a miscarriage, abortion or term delivery (vaginal or caesarean section). Surgery involving the uterine cavity is also sometimes needed for correcting certain uterine malformations particularly, for restarting fertility.

Surgery that involves the uterine cavity is commonly performed from within the uterine cavity itself through surgical hysteroscopy or alternatively, by an abdominal approach. The latter is performed either by laparoscopy or regular laparotomy. In these cases, the uterine cavity may have to be entered if the pathology that needs removal—in general, fibroids—reaches inside the uterine cavity itself.

Surgery of the uterine cavity, by hysteroscopy or abdominal surgery extending to the cavity, carries the risk of developing scar tissue—synechiae—that can cause infertility, pain or other issues or problems associated with such post-surgical developments. Such surgery therefore warrants specific measures to prevent the development of abnormal scarring or synechia formation.

The lining of the uterine cavity—the endometrium—is an extremely delicate structure. It is meant to accept the attachment and development of the embryo at the appropriate time and permit pregnancy to occur. The lining of the endometrium is constituted basically of two types of cell systems, the hormone-sensitive glandular cells and the support tissue constituted of fibroblasts. The former grows primarily under the influence of hormones (mainly estrogens), while the latter, being the support tissue, also responds to inflammatory stimuli, including as caused by surgery that involves the endometrium.

The glandular epithelium of the endometrium is sensitive to hormonal effects. It develops under the influence of estrogen (proliferation of cells resulting in increased thickness) during the first half of the menstrual cycle. During the second half of the menstrual cycle, the lining of the endometrium undergoes sets of sequential transformation under the influence primarily of progesterone. These transformations culminate on the 6^{th} to 7^{th} day of exposure to progesterone in a state of receptivity to embryo implantation or ‘window of receptivity’. The support tissue of the endometrium made of fibroblasts is meant to only provide the necessary environment for the development of the glandular epithelium. In pathological conditions however, the support tissue can overgrow the glandular epithelium and therefore create pathological scarring, often called uterine synechiae, and cause infertility.

In the aftermath of uterine surgery, it is feared that the support tissue—fibroblasts—stimulated by post-surgical inflammation overgrows the hormone responsive cells. Contact with raw surfaces, as it occurs after intra-uterine surgery, will also promote the growth of the support tissue. This will likely result in scar formation that reach between areas of opposing sides of the uterine cavity. The scarring process taking place can cause the formation of synechiae, which are fibrous bridges extending between opposing areas of the uterine mucosa. Ultimately, this may permanently alter receptivity to embryo implantation by the development of abnormal scars or synechiae. As the uterine cavity is normally empty, it said to be virtual, its aspects are normally applied against another. In case of inflammation, as after surgery and/or infection, pathological scarring may develop across the cavity reaching from one side to the other.

Surgery that involves the lining of the uterine cavity constitutes a primary stimulant for the development of pathological scarring in the uterine cavity, or synechiae. The therapeutic strategy for preventing this occurrence after surgery is a simple 2-tier process: (i) to favor the development of hormonally sensitive cells—the glandular epithelium—so that it develops before the support tissue (fibroblasts) grows pathological scars; and (ii) to limit contact between uterine surfaces of opposing sides of the cavity that were denuded by surgery. It is indeed from the raw surfaces that pathological scarring across the cavity has greater chance to develop.

Practically, the only measures taken today for preventing contact between the walls of the uterine cavity consist in placing an intra uterine device (IUD) in the cavity. IUDs which were conceived for contraception are far from ideal in this task. Indeed by being a foreign body, an IUD may itself stimulate the growth of the support tissue. This explains that IUDs are not always placed after uterine surgery, with surgeons each time weighing the pros and cons of using an IUD postoperatively. And aside from IUDs, there was heretofore no practical option for preventing post surgical scarring inside the uterine cavity.

“Scarring of the uterus” consists in the formation of cicatrix tissue inside the uterine cavity. This process is also known as Asherman Syndrome. Normally the uterus is a virtual cavity whose walls are gently and freely applied against one another, leaving in between a film of fluid, but no connective tissue. In certain circumstances, mainly related to inflammatory and/or infectious processes often the result of past uterine surgery, fibrous tissue (“scar”) may develop between the walls of the uterine cavity. This reactive process results in the formation of fibrous bridges between the uterine walls that are the “scars” feared in the uterine cavity. These scars have various degrees of extension—ranging from isolated bridges to a total adhesion of the uterine walls—and different consistency, ranging from flimsy to highly fibrous and rigid.

Prevention of uterine scarring primarily consists in adhering to “good practice” measures when performing common gynecological procedures that involve the uterus. These aim at avoiding chronic uterine infection and inflammation and the possible retention of products of conception following curettages. Such good practice measures will include but are not limited to (i) post D&C ultrasounds for early detection of uterine retention; and (ii) prompt measures—antibiotics followed by repeat D&C—in case of uterine infection.

Other preventive measures proposed for avoiding scar formation consist in inserting a foreign instrument to act as a foreign body in the uterine cavity for avoiding contact between the two uterine walls. This is typically used at the end of certain surgical procedures, for example, the resection of a uterine septum—a procedure known to carry a high risk of scar formation.

The foreign body used for temporarily separating the uterine walls and preventing scar formation are typically IUD and pediatric size Foley catheters. Foreign bodies are used during the normal tissue scarring time, typically 2 weeks, but often extended for longer periods of time (up to 6-8 weeks) under the perception that it might help. These foreign bodies used for preventing scar tissue formation are the only ones available, but are far from ideal. IUDs, the most commonly used instrument for preventing scar formation, do not fully prevent contact between the uterine walls, which still occurs in between the arms of the coil. Moreover, in contrast to helping by separating the uterine walls, the foreign body nature of IUDs is itself a cause of scar tissue formation.

The improper character of current measures taken for prevention of scar tissue formation. IUDs are not designed for that purpose, and their drawbacks have resulted in their inconsistent use. Often, the insertion of intra-uterine instruments is undertaken on the occasion of second (repeat) surgery aimed at removing some already existing scars. These latter procedures are known to leave large areas of raw tissue, where scars were cut, and thus are prone to the recurrence of scar formation.

Clinically, uterine scarring is suspected when changes in bleeding pattern (decrease and/or painful menses) or lack of bleeding is observed following uterine manipulation or surgery or documented uterine infection (endometritis). Uterine scarring enters in the differential diagnosis of all cases of secondary loss of menses (amenorrhea) or infertility.

Intrauterine scarring eludes detection by regular ultrasound imaging. Identifying uterine scarring requires distending the uterine cavity. This is primarily achieved by hysteroscopy (office or diagnostic or surgical) which directly identifies the fibrous tissue connecting the uterine walls. Likewise, hysterosonography will identify pathological filling defects. Alternatively, uterine scarring can be identified on hysterosalpingography (HSG), where scar areas appear as lacunae that are not filled by dye. MRI (Magnetic Resonance Imaging), similar to ultrasounds, will not detect uterine scars as long as the uterine cavity is not distended.

Similar uses of the formulations would benefit procedures and uses involving other body or organ cavities, or other sites on or in the patient's body, to help prevent undesired scarring or otherwise to help facilitate proper and prompt healing. In certain joints for example, surgery for replacing or repairing elements in that joint—including by attempts at regenerating the smooth surface of cartilages through the instillation of stem cell preparations—might benefit from a better consolidation while keeping the healing parts separated from each other during the healing process.

Phase Shifting Gel for Extended Local Therapy to the Uterus

Certain medications may be correct uterine conditions such as notably, increased contractility, inflammation and/or infection. This may be notably encountered in case of premature contraction during pregnancy—premature labor—and in inflammatory conditions such as notably, endometriosis and or adenomyosis and or endometritis (acute and/or chronic sub acute). Systematic treatment of these conditions may be poorly efficient and or associated with side effects. Conversely, local treatment may be short-lived because being too rapidly expelled. Hence a phase-shifting product might enhance and/or prolong such treatment while minimizing side effect due to the local effect.

Such treatments might include:
1. Utero relaxing products for decreasing uterine contraction such as for example in case of premature labor, or prior to a uterine procedure that causes a lasting discomfort such as for example a hysteron salpingogram
2. Utero-contracting substances when uterine contractions are desired as for example for expelling the product of conception after an incomplete miscarriage or for inducing labor
3. Anti infectious and/or anti-inflammatory and/or hormonal modulator substance (including selective progesterone and/or estrogen modulators) for treating uterine condition such notably, endometritis (acute or chronic), endometriosis and or adenomyosis.

Delivery of Treating Agents Within Body or Organ Cavities or Elsewhere

The usefulness of a phase-shifting media to deliver treating agents to various sites within the body becomes apparent when taking into account the ability to adjust the time the media remains in one phase prior to shifting to another. For example, a treating agent can be maintained in a cavity or on a body surface (without physically attaching to, or causing or allowing any solid material to contact, any tissue within the cavity or to on the surface) for a period long enough to impart the desired effect in the cavity or to the surface, then be eliminated from the cavity or off the surface via its phase-shifting properties.

Use For Burns, Wounds, Other Injuries

In case of burns and other extended injuries, the raw and inflamed surface can extend over a large surface. In this case—particularly in case of burn—fluid and electrolytes and proteins can be lost in massive quantities over time. Covering tissue can at times aggravate the situation by absorbing out even more amounts of fluid. There is at this time no ideal way to prevent this possibly massive loss of fluid, proteins and electrolytes. The phase shifting formulation offers a protective barrier of a new type. By not mixing with blood and other bodily fluid, the formulation helps prevent or reduce the constant oozing of fluid and blood constituents such as proteins that can prove critical to the healing process. Such use therefore offers an improved way of covering extended wounds for facilitating the healing process while preventing the loss of fluid and protein in the meantime.

Practically, the substance could be applied over the entire wound using a system adhering to the skin only outside the periphery of the wound and allowing its application under a covering through a one-way valve system. The amount administered would be adjusted to the size of the wound and system used, so that the covering of the system would be lifted by the formulation and not come into direct contact with the wound. The substance so applied could also serve for holding in place graft fragments or colonies of stem cells—layered over the raw area or placed in small clumps—for the time needed so that they attach to the underlying tissue, while preventing during that time course the loss of fluid and proteins that normally takes place in such cases.

The formulation used for such purposes also could be mixed with anti-infective, anti-inflammatory and/or anti-rejection agents, or any type of growth promoting factor that would enhance or facilitate the healing process.

Non-Medical Uses

For example, such formulations may prove useful in cosmetic procedures such as protecting the scalp or facial skin during hair bleaching and coloring procedures; providing protection during tanning in the sun or tanning booths; promoting healing of the site of a fresh tattoo on the body; and maintaining contact with skin or hair for an extended period of time, such as with moisturizing or regenerative agents overnight. The substance could also serve for offering an extended efficacy of insect repellents, allowing limited need for re-dosing and thereby reducing the overall exposure to the repellent. Alternatively, the substance could serve as an inert protective layer to be applied on the skin before using a mosquito or other insect repellent, maintaining the efficacy of the repellent while protecting the skin or any other mucosa from the toxicity or other effects of the repellant.

### SUMMARY OF THE INVENTION

The present invention relates to compositions and methods for separating the different aspects of the uterine cavity, or other body cavities or tissues, typically for a controlled period of time, as for example 2-4 weeks, after surgery and for preventing post-surgical scarring. In the uterus, this ultimately prevents the classical complications, including uterine synechiae, and the ensuing infertility. Other than the uterus, this prevents similar complications inherent to the particular site of surgery.

The composition of the present invention is to be applied after surgery, such as uterine surgery. The composition will consist as a gel or a semi-solid substance that will keep the uterine wall separated for a controlled period of time set to match the time needed so that the normal lining develops while scarring is prevented after surgery. The composition uses phase-shifting properties adapted for the specific needs encountered after the particular surgery. After uterine surgery, for example, about 2-20 ml of the composition will be applied at the end of the surgical procedures (quantity depending on uterine size and the nature of surgery performed), using a catheter. The composition will retain its ability to keep the uterine tissues separated, usually for at least about 10-20 days, but in some cases for up to 8 weeks, the time taken for the glandular epithelium to develop under the influence of estrogen (E2). Approximately 10-20 days after surgery, possibly based on ultrasound appearance or other parameters, doctors will make the decision that healing has sufficiently progressed and so the separation of uterine walls created by the composition is no longer needed.

Further trials will fine tune the optimal duration, which may be different for different indications and/or clinical circumstances. At this point, a product may be instilled in the uterine cavity for liquefying the gel-like composition, which will be naturally expelled. Examples of such products may include, but are not limited to, salts, pH modifying substances, chelating substances, substances which can causes exothermic or endothermic reactions, temperature modifying preparations or preparations which can physically displace the composition. These preparations may be instilled in forms such as, but not limited to, solutions or suspensions.

The composition, often but not always in the form of an inert gel or gel-like substance, will not act as a foreign body, and thus would not be prone to creating an inflammatory reaction, as is the case today with the existing measures (IUD, Foley catheter, etc.). Furthermore, the composition is meant to permit a total separation of the uterine walls from the lower end (inner os of the cervix) to the upper end (fundus) of the uterine cavity. Finally the duration of application of the composition can vary from 1 day to 8 weeks as the removal process (by liquefaction) is totally atraumatic. This is clearly different from IUDs where the pulling required for removal is seen as possibly causing irritation.

The composition remains applied to the uterine walls, separating them for the duration of choice, usually a time of between 1-8 weeks. This offers novel possibilities for applying various locally effective medications, anti-inflammatory, anti-infectives, or diagnostic agents, for extended periods, which could not be done before.

During scar prevention by the composition, ultrasound examination can allow visualization to confirm that the needed slight uterine distension is maintained. If needed, a new administration of the composition could be used to extend the duration of treatment and/or to further maintain wall separation.

Prior to injecting the composition in the uterine cavity, various therapeutic products can be applied locally in the uterine cavity such as anti-inflammatory and healing promoting substances, to further reduce the risk of scarring. In some embodiments, the composition further includes such substances within its formulation. The substances that can be used locally before applying, or included within, the composition for maintaining the desired separation between the opposing aspects of the uterine cavity include, but are not limited to: corticosteroids, estrogenic hormones, progesteronic hormones, NSAID preparations, protectorants, various anti-oxidants and/or all active preparations that are commonly found in balms, creams, gels, lotions and ointments used in other healing and/or as topical anti-inflammatory preparations. Possible substances further include, but are not limited to, aspirin, salicylic acid derivatives, zinc oxide, vitamins A, C or E (and derivatives), tissue growth factors, stem cell derived tissue grafts, and emollients, all used in concentration ranges commonly used in practice. The composition would help maintaining contact between such substances and the endometrial mucosa. Endometrial cells with stem-cell like pluri-potent properties could also be placed over the surfaces left raw by surgery. Endometrial cells from the basal layer display pluri-potent properties that make them true stem cells. Such cells collected from a healthy area of the endometrium could be replaced in thin layer over the raw surfaces left after uterine surgery.

This process could take place directly with cells collected and replaced elsewhere during the same procedure. Alternatively, endometrial cells from healthy areas could be obtained before the scheduled uterine surgery and made to multiply in vitro in order to obtain a sufficient amount for covering the raw surfaces at the end of the uterine surgery. Such cells could either be simply layered over the desired area where regeneration of a normal endometrium is desired or held in some sort of mesh designed to play a holding role or scaffold. The latter can be made of persistent or resorbing material.

But cells that would be simply layered on the damaged endometrial surface would be washed away before having the chance of regenerating a proper uterine lining. On the contrary, the composition, applied just after the cells are layered, would help keep them in place for the desired duration. By providing a practical way of holding cells in place, the composition could therefore make cell therapy possible for the regeneration of damaged endometrium. In that, the composition could be crucial for preserving or restoring a functional lining of the uterine cavity. Ultimately therefore, the invention could markedly change the prospect of pregnancy after uterine surgery.

The skills and knowledge base to produce effective anti-inflammatory and cell therapy formulations is standard among those skilled in the art. In particular, the preparations susceptible of helping the healing process of the endometrium after surgery of the endometrium have known application to mucosal and epithelial surfaces. Currently however the use of such formulations and cell therapy is simply impractical. The invention, which makes these options possible, allows for the timing necessary to have these formulations perform as needed to offer the desired results.

Prevention of Scarring, Facilitation of Healing in Other Settings

Similar benefits can be achieved by using the formulations to help minimize scarring elsewhere in the body, as well. At the same time, the formulations may help facilitate healing, in different settings. For example, after certain urological procedures, the formulation may help facilitate healing without excessive scarring. In cases where tissue is excised, healing may be enhanced by promoting setting and healing of the wound without excessive scar formation.

In other surgeries performed in closed body or organ cavities, such as encountered in urology or while performing reconstructive surgery in joints, it might be helpful to keep such a cavity slightly distended during the healing process. This would facilitate the natural healing process so that it can take place undisrupted by the natural tendency that raw surfaces have to develop pathological scarring—adhesions—when they come in contact with each other. Upon advancement of the natural healing—when the raw surfaces have regenerated their natural protective covering—the formulation may be removed to allow completion of the healing process. The removal of the formulation can be done in different manners using the phase shifting properties of the substance. In cavities having a natural opening to the outside—as the uterus for example—a second product transforming the gel into liquid would allow natural expulsion. In other enclosed cavities, as in joints, for example, transforming the formulation into liquid phase would allow for an easy removal by aspiration (which is not feasible for a regular gel).

Delivery of Treating Agents Within Body or Organ Cavities

The phase-shifting formulations provide the ability to deliver drugs and keep them in contact within a cavity for an extended period of time. This would be especially useful in cavities which do not typically have any or much significant movement through them, or cavities that are essentially a ‘dead end,’ such as the uterus or bladder. Such agent could include anti-infectives, anti-inflammatories, hormonal agent, and agents to promote or retard healing, as may be deemed suitable by the practitioner. Delivery of the active agent could be either before the phase-shifting formulation is placed in the body cavity, or could be contained by the formulation itself.

Delivery of Treating Agents to Sites Other Than Body or Organ Cavities

The formulations can also easily be designed for delivery to other body parts besides cavities. Delivery of treating agents over time without any real structure contacting the damaged or injured tissue will frequently help facilitate proper and prompt healing, often helping to avoid common issues otherwise of concern.

Burns and Other Wounds

Through the use of the formulation as a distending medium in the uterine cavity for performing hysteroscopic explorations, we came to observe that blood does not mix with the viscous formulation. Thus, we realized that the formulation in its first, viscous stage prevents blood from flowing from the uterine wall.

We realized accordingly that this unexpected effect provides usefulness of the formulation for preventing bleeding and oozing from wounds, and thereby helping to prevent loss of protein extrudate, promoting and enhancing faster and more complete healing.

Non-Medical Uses

For example, such formulations may prove useful in cosmetic procedures such as protecting the scalp or facial skin during hair bleaching and coloring procedures; providing protection during tanning in the sun or tanning booths; promoting healing of the site of a fresh tattoo on the body; and maintaining contact with skin or hair for an extended period of time, such as with moisturizing or regenerative agents overnight.

### DETAILED DESCRIPTION OF THE INVENTION

Surgical procedures affecting the uterine cavity—performed by endo-uterine procedures or hysteroscopy or abdominal approach (laparotomy or laparoscopy)—carry the risk of causing scar tissue formation—or synechia(e). These inflammatory reactions are capable of interfering with further fertility by altering uterine (or endometrial) receptivity to embryo implantation. The lying of the uterus or endometrium is constituted of distinct tissues. First there is the noble or functional tissue defined as the epithelium that carries its functional role of enabling embryo implantation and pregnancy development. Second there is—as in all organs—the supportive or conjunctive tissue made essentially of fibroblast cells.

Proliferation or ‘development’ of the endometrium is regulated by hormones, which exert their physiological effects in a sequential manner. First, estrogens induce full endometrial development or ‘estrogen priming’ that enables its subsequent response to the second hormone, progesterone, in a 2-3 weeks time. Second, progesterone—typically produced after ovulation or administered secondarily in a hormone-priming regimen used in assisted reproduction—induces the sequences of transformation of the endometrium needed for embryo implantation.

After uterine surgery for pathologies such as the removal of polyps or fibroids, the support connective tissue and its fibroblast are stimulated to grow by the inflammatory nature of the wound created. This may lead to such a proliferation of fibroblast that scar tissue develops and join the opposing surfaces of the lining of the uterus thereby obstructing totally of partially the uterine cavity and impending the proper development of the embryo. This inflammatory process of scar tissue or siniechia(e) formation—Ascherman syndrome—may lead to either lack of embryo implantation and infertility or repeated miscarriages.

In general, the walls of the uterine cavity will benefit from staying separated from one another in order to prevent scar formation for up to 8 weeks or so following surgery involving the uterine cavity. Two weeks are usually sufficient to allow for the development of the endometrial mucosa and its secretion which will constitute the best protection for scar formation. In certain circumstances however, clinicians may be inclined to keep the uterine walls separated for longer periods of time, as for example 6-8 weeks. It would be rare that an even longer time of separation of uterine walls will appear beneficial for preventing scar formation, but such longer time periods would be readily available with the right formulations.

Whether or not the addition of an agent to liquefy the composition to facilitate removal once the healing has taken place is needed is, again, formulation and time dependent. In most embodiments, a second agent will be instilled which will cause the composition to change into a state which is easily removed/expelled from the cavity. However, in some embodiments, the formulation is formulated with phase-shifting properties that will be triggered automatically, such as over time, and will be removed from the body without the addition of a second agent. This, too, is subject to experimentation.

Different constituents and combinations of constituents can provide the desired qualities needed for the development of products that can be used for the invention. These fall in the range of substances which form thickened, semi-solid or even solid compounds.

Ingredients useful to formulate compositions of the invention include, but are not limited to, celluloses, carbomers, starches, polxamers and colloidal clays. One of skill in the art will be able to use these or similar ingredients to make formulations which will remain on tissue surfaces and retain their structural integrity. The formulator will be able to select a “trigger” that will cause the composition to lose its integrity and be able to be removed from the tissue surface.

The optimal time frame during which the products used in the compositions of the invention need to remain in contact with the tissue, or in the cavity, depends on the specific circumstances of the particular patient. In fact, depending upon the specific usage the time may vary significantly. The medical practitioner will readily be able to determine the appropriate timeframe for the particular patient and situation. The formulator will readily be able to prepare a formulation with the timing characteristics specified by the medical practitioner, and including any additives to be mixed in or pre-applied. This variance may also be directly related to the formulation used. Constituents, combinations and ratios are routine in the art.

Formulations

The compositions of the invention can be made using ingredients that provide an inert, mostly impermeable formulation that will remain on tissue surfaces. This can be achieved using most gel-forming ingredients, including, but not limited to polymers (such as celluloses (including without limitation methylcellulose, ethylcellulose, hydroxyethyl cellulose, ethyl hydroxyethyl cellulose, hydroxypropyl cellulose, hydroxypropyl methylcellulose, and carboxymethylcellulose), carbomers, gelatin, agar, pectin, starches, high molecular weight polyethylene glycol), copolymers (such as, for example, poloxamers) and colloidal clays (such as, for example, bentonite or tragacanth).

The formulation could be made by one of skill in the art to remain intact until a “trigger” causes the structure to lose its integrity and dissociate from the tissues and be expelled from the body. These formulations differ from the formulations used in U.S. Pat. No. 7,727,155, which is hereby incorporated in its entirety, in that they are inherently more stable structurally and retain viscosity for days and weeks instead of minutes. In addition, the composition will have a greater tendency to remain on tissue surface in comparison to the formulations in U.S. Pat. No. 7,727,155.

Triggers that may be used with the compositions of the invention include, but are not limited to, preparations which can modify the composition's structure by changing salt concentrations (either higher or lower) to control rate and timing of when a formulation would change phase chelating certain molecules, or causing exothermic or endothermic reactions, changing the temperature of the composition directly, changing pH of the composition or the environment, physically disrupting the composition's formulation or displacing the composition's formulation from the tissues, so long as the change to the formulation results in the desired change in viscosity or liquefaction, or otherwise causing the formulation to lose viscosity and be more easily removed or expelled from the site of administration.

While the uterine cavity is the primary area in which the use of the invention is foreseen, compositions of the invention can also be used in other organs and territories. These other applications include, for example, in the Fallopian tubes or the urethra, or in the bladder.

In the Fallopian tubes, application of a composition of the invention at the end of surgery would maintain separation of the tube while the healing process takes place. A certain time later, for examples up to 2-4 weeks later, the product would liquefy, either subsequent to the nature of the formulation itself, or by instilling a second product through the uterine opening of each tube.

Allowing the walls of the Fallopian tubes to freely contact only after proper healing has taken place would likely favor better preservation of tubal functionality. Hence, the proposed product would likely decrease the risk of the complication of tubal surgery, such as infertility by tubal occlusion and/or alteration and ectopic pregnancy and the ensuing new tubal surgery. In the ureter or male urethra, the product inserted around a stent would prevent direct contact between tissue (the urinary epithelium) and the stent, and possibly allow for removal of the stent with an opening for urine flow remaining within the product itself. A liquefying second product could be administered by retrograde injection or by urinary excretion. Likewise, benefit could be obtained for surgery performed in or on any organ, body cavity, or tissue where benefit might be gained from keeping the surfaces separated after surgery, as for example in otic, nasal, orthopedic, neurosurgery or other surgical procedures.

The instant invention would be useful during various types of medical procedures, whether for observational, diagnostic, treatment, or other purpose. These include, without limitation, procedures such as x-rays, ultrasound, CT scans, MRI, HSG, or endoscopy.

The problems encountered for holding a contrast medium for imaging procedures such as MR imaging, CT-scans etc. could be handled by the time-controlled phase shifting properties of the substance. In ultrasound imaging techniques now available, constant infusion of a contrast fluid—while cumbersome—can be performed. In other imaging techniques, infusion during the procedure is not possible, without use of a phase-shifting formulation. The phase shifting properties would make the use of intrauterine contrast substances possible while allowing for their removal afterwards through the phase shifting properties of the substance and utilizing several systems and/or methods for controlling the phase shifting.

Methods of Administration

The necessary amount of the composition, about 2-20 ml, is instilled in the uterine cavity using a syringe attached to a plastic catheter that is passed through the cervical canal. Upon instillation the composition has a gel-like consistency. The exact degree of uterine distension can be verified using ultrasound either during the process of administration of the composition or afterwards. People skilled in the art of practicing uterine surgery and uterine manipulations could determine the best degree of distension desired, possibly adjusting the parameters according to the different diagnoses.

Alternatively, the composition could be administered directly through an injecting port permanently or temporarily attached to a hysteroscope that was used during surgery and/or the layering of anti-inflammatory substances and/or layers of endometrial or stem cells. One of skill in the art would be able to manipulate the hysteroscope to apply the composition.

Some small amounts (3-5 cc) of phase-shifting formulation would be instilled in the uterine cavity at the end of surgical procedure on the uterine cavity—from the inside (during a hysteroscopy) or the outside of the uterus (during laparotomy or laparoscopy). The phase shifting formulation would serve for slightly distending the opposing raw surfaces of the uterine cavity in order to avoid contact between the surfaces for the duration of the healing process and development of the noble tissue—the endometrium. This process typically takes 2-3 weeks. At the end of this time interval, the distending formulation would liquefy—either spontaneously by a time-set phase shifting process or through the instillation of a few CC of a second liquefying solution. Once liquefied the few CCs of formulation would be expelled naturally and easily.

The phase shifting formulation used for temporarily distending the uterine cavity could be neutral or combined with different active substances designed for preventing inflammation and/or infection development, as deemed necessary.

For the purpose of treating endometrial lesions—adhesions, area of dysfunctional disruption non-responding to hormonal treatment—with stem cell therapy. The phase-shifting formulation will facilitate the positioning and keeping in place of stem cell preparations so that the uterine walls are kept separated during the process in order to prevent the development of inflammation. Stem cells simply applied over a raw or functionally disrupted area would otherwise require surgical removal—peeling—of prior dysfunctional scarring in order to re-colonize the disrupted areas and generate a new functional tissue emanating from the population of stem cells. Taking place in an area purposely rendered raw could again generate a pathological dysfunctional scarring destroying the stem cells before they could help the physiological healing. The substance would offer the advantage of an inert covering of the area where healing is expected to take place from the stem cells layered there. Tissue protection by the substance would be required for the duration of the physiological healing, a time course that vary depending on the tissue but would generally range from 10 to 20 days.

The phase-shifting formulation also could be used in conjunction with various treating agents, such as different products exerting properties that facilitate the development of stem cells or on the contrary, prevent the growth of non-specific fibroblast that could prevent stem cell development.

Preventing Scarring Elsewhere in the Body

Example: Urology

A surgical wound in urology might benefit from a phase shifting gel that would separate the raw areas of the surgical wound from the opposite surfaces for the time of the healing for preventing scar tissue formation. For example, surgical resection of benign or cancer lesions of the prostate and other parts of the urological canal could benefit from being kept separated from the opposite surface for the time of healing, by helping to prevent adherence, undesired scarring, or accelerated scarring. And again, anti-infective agents or other treating agents may also be included in such formulations, to simultaneously provide multiple benefits.

Facilitating Healing

These formulations may provide advantages when used for facilitating healing following urological procedures. For example, after certain urological procedures, the formulation may help facilitate healing and appropriate scarring. In cases where tissue is excised, healing may be enhanced by promoting limited initial scar formation.

During the time that the formulation would serve for separating the raw surfaces or the urological procedure, a supra-pubic urinary catheter would serve for temporarily redirecting the flow of urine.

Delivery of Treating Agents Within Body or Organ Cavities or Elsewhere

The usefulness of a phase-shifting media to deliver treating agents to various sites within the body becomes apparent when taking into account the ability to adjust the time the media remains in one phase prior to shifting to another. For example, a treating agent can be maintained in a cavity or on a body surface (without physically attaching to, or causing or allowing any solid material to contact, any tissue within the cavity or to on the surface) for a period long enough to impart the desired effect in the cavity or to the surface, then be eliminated from the cavity or off the surface via its phase-shifting properties. One or more treating agents could be delivered within such cavities during a medical procedure by adding the treating agent to the formulation used during the procedure, or they could be separately or again delivered, after a procedure or otherwise, in a similar formulation that could be designed to remain in place for a longer period of time—up to weeks or more, as appropriate.

Non-Medical Uses

For example, such formulations may prove useful in cosmetic procedures such as protecting the scalp or facial skin during hair bleaching and coloring procedures; providing protection during tanning in the sun or tanning booths; promoting healing of the site of a fresh tattoo on the body; use with agents such as insect repellants, to provide lasting protection that is easily removed when desired; and maintaining contact with skin or hair for an extended period of time, such as with moisturizing or regenerative agents overnight. For certain uses, such as potentially with insect repellants, the formulation could be designed to help protect the user from the insect repellant, while maintaining the repellant's efficacy, by forming a barrier between the skin of the user and the repellant.

Suitable Treating Agents

Active ingredients suitable for use in or with the present invention include, but are by no means limited to: (1) glycoproteins, such as follicle-stimulating hormone (FSH), luteinizing hormone (LH), human chorionic gonadotropin (HCG), thyroid-stimulating hormone (TSH), and the like; (2) proteins, such as GnRH (agonist and antagonist), desmopressin, oxytocin analogs, insulin analogs, TRH analogs, somatostatin analogs, tissue plaminogen activator (TPA), growth hormone releasing hormone (GHRH), corticotropin-releasing hormone analogs (CRH analogs), and the like; (3) sex hormones, such as estradiol, testosterone, progesterone, other estrogenic and progestogenic compounds, and the like; (4) anti-hormones and selective estrogen and progestin receptor modulators, such as tamoxifen, mifepristone, raloxifene, and the like; (5) nitrates, such as nitroglycerin, isosorbide, erythrityl tetranitrate, pentaerythritol tetranitrate, and the like; (6) beta-agonists, such as terbutaline, albuterol, pirbuterol, bitolterol, ritodrine, and the like; (7) beta-antagonists, such as propranolol, metoprolol, nadolol, atenolol, timolol, esmolol, pindolol, acebutolol, labetalol, and the like; (8) opioids, such as morphine, hydromorphone, oxymorphone, codeine, hydrocodone, oxycodone, levorphanol, levallorphan, buprenorphine, fentanyl, nalbuphine, butorphanol, pentazocine, and the like; (9) opioids-antagonists, such as naloxone, nalmefene, and the like; (10) antidepressants, such as amitriptyline, amoxapine, desipramine, doxepin, imipramine, maprotilen, nortriptyline, protripyline, trimipramine, fluoxetine, trazodone, and the like; (11) HMG CoA reductase inhibitors, such as lovastatin, mevastatin, simvastatin, pravastatin, atorvastatin, and the like; (12) antihistamines, such as loratadine, chlorpheniramine maleate, brompheniramine maleate, diphenhydramine, dimenhydrinate, carbinoxamine, promethazine, tripelannamine, and the like; (13) ACE inhibitors, such as captopril, enalapril, lisinopril, and the like; (14) prostaglandins, are a class of naturally occurring chemically related, long-chain hydroxy fatty acids, such as prostaglandin E₂ (“PGE₂”), PGE₁, PGA₁, PGB₁, PGF_{1α}, 19-hydroxy-PGA₁, 19-hydroxy-PGB₁, PGA₂, PGB₂, 19-hydroxy-PGA₂, 19-hydroxy-PGB₂, PGE₃, PGF_{3α}; semisynthetic or synthetic derivatives of natural prostaglandins, including mioprostol, carboprost tromethamine, dinoprost tromethamine, dinoprostone, lipoprost, gemeprost, metenoprost, sulprostone and tiaprost; analogues thereof and the like; (15) non-steroidal anti-inflammatory drugs (NSAIDS), such as diclofenac, etodolac, fenoprofen, lurbiprofen, ibuprofen, indomethacin, ketoprofen, ketorolac, meclofenamate, fenamic acid, meloxicam, nabumetone, naproxin, oxaprozin, piroxicam, sulindac, tolmetin, and the like; (16) anti-infectives; (17) anesthetics, such as lidocaine, cocaine, chloroprocaine, tetracaine, prilocaine, mepivacaine, buipivacaine, levobupivacaine, articaine, ropivacaine, phenol, benzocaine, pramoxine, dyclonine, etidocaine, procaine, proparacaine, dibucaine, and pramoxine; (18) immune system modifiers such as imiquimod and the like; (19) muscarinic agonists and antagonists such as bethanecol and oxybutinyn and the like; (20) anti-neoplastic agents including alkylating agents such as melphalan, antimetabolites such as fluorouracil, and natural products such as vinca alkaloids and bleomycin as well as agents such as cisplatin and the like; (21) vitamin K; (22) ondansetron; (23) levocarnitine; (24) anti-fungals; (25) carbamide peroxide; (26) dopamine antagonists (bromocriptine); (27) bisphosphonates: (28) nicotine; (29) anti-virals (acyclovir); (30) anti-diabetagenics (metformin); (31) peptides (octreatide, desmopressin, GNRH, other proteins); (32) insulin; (33) anti-Parkinson agents (levadopa); (34) low molecular weight heparins; (35) antimicrobials such as metronidazole and the like; (36) anti-cancer agents, such as tumor specific agents or other agents; and (37) anti-infective compounds such as heavy metals, salts, and radio-active substances. Accordingly, one of ordinary skill in the art will appreciate that formulations according to the invention may be used with a wide variety of active ingredients to treat a wide variety of conditions, and that the ingredients could be placed separately, before the gel formulation, or they could be included within the gel formulation itself.

Burns, Wounds

Blood and other bodily secretions ooze or flow from wounds, including serious burns. The formulation typically will prevent the oozing of such fluids from the wound and/or raw area by not mixing with the fluid, and thus retaining critical protein exudate in the wound. This property could be used for diminishing inflammation, accelerate healing and provide a nicer less destructive and/or disharmonious scar. The retention property is lost as soon as the viscosity decreases and the formulation liquefies. The formulation could be applied on the raw surface and/or open wound as it is in an inert sterile form or combined with active substances such as anti-inflammatory and/or antibiotics and/or other substances capable of ameliorate the healing process. Similarly, the active substance could be, for example, sprayed on the wound, or otherwise administered before the phase-shifting formulation is put in place.

Thus the phase-shifting formulation provides potential use for protecting such injuries without the normal detrimental effects of using fabric bandages. Instead, the formulation enables preparation of a new skin bandage with no material touching the skin for extended wounds like burns, or for sensitive areas, like the face.

For example, there currently is no real way to cover a wound without any material touching the surface of the wound, or while at least preventing the oozing of fluid and blood to attach to the covering. Removing or changing such coverings without disturbing the wound is nearly impossible, and inevitably causes significant pain, too, as well as delaying the healing process.

Use of these formulations may provide a “bandage” or covering without direct contact with the healing wound by the covering fabric. Using the formulation to separate the bandage from the underlying wound would greatly enhance the healing process, reducing discomfort for the patient. Further, with certain injuries such as extensive serious burns, the formulation may help prevent fluid losses and maintain crucial factors such as coagulation factors in the patient's bodily fluids.

For example, a large bandage with a valve system could allow changing or refreshing the formulation by first liquefying the portion already covering the wound, and then gently removing it or allowing it to seep away while a fresh portion of formulation is administered. Such periodic refreshing of the formulation would allow removal of cell debris and/or delivering new dosing of an active substance—any desirable active agents could be included in or along with the formulation, for any or all of the administrations.

One possible embodiment would provide a constant or pulsatile flow of formulation periodically, allowing periodic phase changes while applying only slight pressure through two valves opening into the bandage system. Such gentle treatment of the wound, combined with preserving more of the patient's natural fluids and agents, should provide a significant advantage in treating certain injuries, especially burns, and most especially large burns.

Another use would be as an emergency application to a burn or wound in the field, pending full medical treatment. Promptly coating and protecting the wounded tissue until full medical attention is available would help to minimize infection, further damage, and fluid loss. The easy removal of the formulation after liquefaction facilitates prompt full medical attention as soon as it is available.

This could be particularly important for covering large burns from which large amounts of fluid rich in protein and other blood constituents are lost daily to the point of making the compensation difficult.

For example, the formulation could be used in combination with a bandage of different size and shape adhering at the periphery under which a suitable formulation would be injected through a valve system opening until sufficient material is present for a) avoiding contact between the damaged surface and the covering, and b) helping retain the blood and fluid and preventing the constant effusion that characterizes large burns.

The formulation could either be neutral or contain any type product deemed helpful for facilitating healing and/or preventing complications such as infections.

The principle described above for offering a formulation coverage or protective layer could apply not only on skin wound as described above but also to protect and help treatment of wounds and/or post traumatic scar tissue of internal organs such as possibly encountered in gynecology (intra-uterine or tubes), urology or ENT (ear, nose and throat) practices.

### Other Medical Uses

Skin Grafts

The formulation virtual barrier could also serve for holding in place fragments of graft tissue and/or stem cell preparations and/or any growth factor and/or anti rejection substance in case of grafting.

Joints

Diagnosis of damages or developing lesions (degenerative or linked or inflammation or cancer process) could be facilitated by the temporary distension of the joint, and delivering formulation, with or without treating agents, to the joint space.

Urology

Certain diagnostic processes (ultrasound, MRI, etc.) looking for urological lesions could be facilitated by generating contrast interphases in between surfaces that are normally collapsed on themselves. Such formulations also could be used in combination with treating agents.

Dental Use

There are potential uses for the formulation during and after dental procedures, too. The formulation could be used to maintain active agents, such as antibiotics, in place, such as after or during an extraction, or a root canal procedure. Use during or after gum surgery similarly could help maintain an appropriate treating agent, like an antibiotic, in place for an extended period of time. Alternatively, the composition could be used to protect soft tissue during use of bleaching agents for whitening teeth, or for administering fluoride treatment to the teeth, or to coat the gums to protect them from sticking to cement or adhesive used during procedures.

Non-Medical Uses

For example, such formulations may prove useful in cosmetic procedures such as protecting the scalp or facial skin during hair bleaching and coloring procedures; providing protection during tanning in the sun or tanning booths; promoting healing of the site of a fresh tattoo on the body; and maintaining contact with skin or hair for an extended period of time, such as with moisturizing or regenerative agents overnight.

Any and all publications and patent applications mentioned in this specification are (1) indicative of the level of skill of those skilled in the art to which this invention pertains, and (2) hereby incorporated by reference to the same extent as if each individual publication or application was specifically and individually incorporated by reference.

It is to be understood that the invention is not to be limited to the exact configuration as illustrated and described herein. Accordingly, all expedient modifications readily attainable by one of ordinary skill in the art from the disclosure set forth herein, or by routine experimentation therefrom, are deemed to be within the spirit and scope of the invention as defined by the appended claims.

1. A phase-shifting formulation for avoiding post-surgical scarring and other adhesions, comprising a suitable gel or semi-solid component in a composition that is placed on a post-surgical tissue surface before, during, or after a surgical procedure, and keeps local tissues separated while allowing healing, and then is modified to facilitate removal from the surgical site.

2. (canceled)

3. The formulation of claim 1 wherein the formulation is removed by use of a trigger that causes the formulation to lose its integrity to facilitate removal from the body cavity or tissue surface.

4. (canceled)

5. (canceled)

6. The formulation according to claim 1 wherein the formulation also delivers or maintains a treating agent or tissue regeneration stimulating agent to or on the tissue to promote healing and/or regeneration.

7. (canceled)

8. (canceled)

9. A method of preventing or minimizing post-surgical scarring or other adhesions at a site that needs to heal post surgery, comprising: preparing a viscosity-shifting composition for use during or after surgical and other medical procedures wherein the composition maintains an initial viscosity or consistency for a time sufficient to maintain the subject body tissue free from contact with other tissue or objects, and the composition subsequently reduces in viscosity or consistency over a relatively short period of time, sufficiently so as to allow the composition to be easily expelled or removed from the body tissue; conducting the surgical or other medical procedure; implacing the composition to coat or cover the site of the procedure that requires healing; allowing the composition to then, after a suitable amount of time, reduce in viscosity or consistency over the relatively short period of time; removing or allowing expulsion of the reduced viscosity or reduced consistency composition from the body tissue; and repeating the implacement of a fresh amount of the composition if and as needed until the risk of scarring or adhesions has decreased significantly.

10. The method of claim 9 wherein at least one further treating agent is applied to the site or included in the viscosity-shifting composition.

11. The method of claim 10 wherein the at least one treating agent includes at least one anti-infective agent.

12. The method of claim 10 wherein the at least one treating agent includes at least one anti-inflammatory agent.

13. A method of treating a part of a patient's body or of preventing or minimizing the development of a condition at a part of a patient's body, comprising: preparing a viscosity-shifting composition wherein the composition maintains an initial viscosity or consistency for a time sufficient to achieve the treatment for at least some time, and the composition subsequently reduces in viscosity or consistency over a relatively short period of time, sufficiently so as to allow the composition to be easily expelled or removed from the body tissue; administering the composition to coat or cover the site of the procedure that requires such prevention or treatment; allowing the composition to remain in place for a suitable amount of time, either once or with sequential administration of appropriate amounts of the formulation as needed, after each prior amount of formulation had reduced in viscosity or consistency over the relatively short period of time; removing or allowing expulsion of each quantity of the formulation at the reduced viscosity or reduced consistency composition from the body tissue; and repeating the administration of a fresh amount of the composition if and as needed until the desired treatment has substantially occurred.

14. The method of claim 13 wherein the treatment is intended to minimize or reduce development of scarring or adhesions after a medical procedure.

15. The method of claim 14 wherein the treatment is applied to one or more of a patient's uterus, fallopian tubes, colon, urethra, or bladder.

16. (canceled)

17. (canceled)

18. (canceled)

19. (canceled)

20. The method of claim 13 wherein the treatment is intended to minimize or prevent infection or inflammation.

21. The method of claim 13 wherein the treatment is intended to protect a wound from infection or from further damage pending medical attention.

22. The method of claim 13 wherein the treatment is intended to maintain a skin graft in place.

23. The method of claim 13 wherein the treatment is intended to facilitate diagnosis of damage or developing lesions in or near a joint of the patient.

24. The method of claim 13 wherein the treatment is intended to be used during a cosmetic procedure.

25. The method of claim 13 wherein the treatment is intended to protect a patient's burns.

26. The method of claim 25 wherein the treatment also is intended to facilitate healing of the burns by protecting from loss of blood and bodily fluids.

27. The method of claim 26 wherein the treatment includes placing additional treating agents to help promote healing of the patient's burns either on the burn or within the viscosity-shifting composition.
